(19) United States
(12) Patent Application Publication
(10) Pub. No.: US 2021/0153546 A1
(43) Pub. Date: May 27, 2021

(54) LIQUID TOBACCO EXTRACT

(71) Applicant: BRITISH AMERICAN TOBACCO (INVESTMENTS) LIMITED, LONDON (GB)

(72) Inventor: Helena DIGARD, London (GB)

(21) Appl. No.: 16/614,119

(22) PCT Filed: May 9, 2018
(86) PCT No.: PCT/EP2018/062123

§ 371 Date: Nov. 15, 2019

(30) Foreign Application Priority Data
May 15, 2017 (GB) 1707769.4

(51) Int. Cl. A24F 40/30; A24F 40/10; A24F 40/20; A24F 40/42; A24B 15/167; A24B 15/24

(52) U.S. Cl. CPC A24F 40/30; A24F 40/10; A24B 15/24; A24F 40/42; A24B 15/167; A24F 40/20

(57) ABSTRACT

Embodiments described herein include a cartridge for use with an apparatus for generating an inhalable medium, the cartridge comprising: a first chamber containing a liquid, the liquid comprising a liquid tobacco extract; a second chamber configured to receive a solid flavor material; and the cartridge being arranged such that in use, the liquid is volatilized to form a vapor or an aerosol, and the vapor or aerosol combines with one or more components of the solid flavor material received in the second chamber to form an inhalable medium, the inhalable medium comprising one or more constituents of the solid flavor material entrained in the vapor or aerosol.

### PRIORITY CLAIM

The present application is a National Phase entry of PCT Application No. PCT/EP2018/062123, filed May 9, 2018, which claims priority from GB Patent Application No. 1707769.4, filed May 15, 2017, which is hereby fully incorporated herein by reference.

### TECHNICAL FIELD

The present invention relates to a liquid tobacco extract and the use of a liquid tobacco extract in a device for generating an inhalable medium.

### BACKGROUND

Smoking articles such as cigarettes, cigars and the like burn tobacco during use to create tobacco smoke. Attempts have been made to provide alternatives to these articles that burn tobacco by creating products that release compounds without burning. Examples of such products are heating devices which release compounds by heating, but not burning, material. The material may be, for example, tobacco or other non-tobacco products, which may or may not contain nicotine.

Electronic cigarettes or “e-cigarettes” are another product that has been formulated as an alternative to combustible products. The known e-cigarette devices do not contain or use tobacco. Rather, these devices contain a volatilizable solution which generates an inhalable aerosol on heating. These solutions may contain aerosol-generating substances. The solution may also contain components of tobacco, such as nicotine and/or flavorings. It is therefore useful to be able to selectively extract tobacco components.

As another example, there are e-cigarette/tobacco heating product hybrid devices, also known as electronic tobacco hybrid devices. These hybrid devices contain a liquid which is vaporized by heating to produce an inhalable vapor or aerosol. The liquid may contain nicotine and/or flavorings and/or aerosol-generating substances. The vapor or aerosol passes through material in the device to entrain one or more constituents in the material to produce the inhaled medium. The material may be for example tobacco or other non-tobacco products, which may or may not contain nicotine.

### SUMMARY

According to a first aspect of the present invention, there is provided a cartridge for use with an apparatus for generating an inhalable medium, the cartridge comprising:

a first chamber containing a liquid, the liquid comprising a liquid tobacco extract;

a second chamber configured to receive a solid flavor material; and

the cartridge being arranged such that in use, the liquid is volatilized to form a vapor or an aerosol, and the vapor or aerosol combines with one or more components of the solid flavor material received in the second chamber to form an inhalable medium, the inhalable medium comprising one or more constituents of the solid flavor material entrained in the vapor or aerosol.

In some cases, the cartridge additionally comprises a channel providing fluid communication between the first chamber and the second chamber, wherein in use, the aerosol or vapor formed by volatilizing the liquid passes through the channel to the second chamber and through the solid flavor material to form the inhalable medium.

In some cases, the second chamber contains the solid flavor material. In some cases, the solid flavor material comprises tobacco.

The liquid tobacco extract may be that obtainable by or obtained by a process comprising (i) extraction of tobacco components from tobacco using a supercritical extraction solvent, and (ii) transfer of the extracted tobacco components into a liquid solvent.

The inventors have found that the use of a tobacco extract as the liquid component in an electronic tobacco hybrid device provides a beneficial organoleptic effect. The tobacco extract contributes to the flavor of the inhaled aerosol. Typically, the liquid tobacco extract delivers the tobacco taste base note, and the solid tobacco portion delivers top notes of the overall sensory experience.

The inventors have also established that the use of a tobacco extract as the liquid component provides longevity of taste. Tobacco flavor is delivered throughout the consumption period. Further, the liquid tobacco extract is a better vehicle for delivery of the taste/flavor of less aromatic tobaccos (e.g. conventional Virginia tobacco), than solid tobacco.

Further, liquid tobacco extracts typically contain nicotine. Pharmaceutical grade nicotine (which is incorporated into typical prior art hybrid liquid solutions) can consequently be employed at lower concentrations and in some cases, need not be employed at all. Pharmaceutical grade nicotine is highly purified and requires significantly more processing than a liquid tobacco extract.

According to a second aspect of the invention, there is provided apparatus for generating an inhalable medium, the apparatus comprising a cartridge according to the first aspect of the invention, and an outlet, the apparatus being configured such that the inhalable medium passes out of the outlet.

The invention also provides apparatus for generating an inhalable medium, the apparatus comprising:

a first chamber containing a liquid, the liquid comprising a liquid tobacco extract;

a second chamber configured to receive a solid flavor material; and an atomizer for volatilizing the liquid in the first chamber; and

an outlet;

the apparatus being configured such that, in use, the liquid is volatilized to form a vapor or an aerosol, and the vapor or aerosol combines with one or more components of the solid flavor material received in the second chamber to form an inhalable medium which passes out of the outlet, the inhalable medium comprising one or more constituents of the solid flavor material entrained in the vapor or aerosol.

In some cases, the apparatus additionally comprises a channel providing fluid communication between the first chamber and the second chamber, wherein in use, the aerosol or vapor formed by volatilizing the liquid passes through the channel to the second chamber and through the solid flavor material to form the inhalable medium.

In some cases, the second chamber contains the solid flavor material. In some cases, the solid flavor material comprises tobacco.

A third aspect of the invention provides a kit of parts, the kit comprising a cartridge according to the first aspect of the invention and a volatilizing device, wherein the cartridge is configured to be operable with the volatilizing device so as to generate an inhalable medium.

A fourth aspect of the invention provides a method of generating an inhalable medium, the method comprising (i) volatilizing a liquid to form a vapor or aerosol, wherein the liquid comprises a liquid tobacco extract, and (ii) combining the vapor or aerosol with components of a solid flavor material to form an inhalable medium, the inhalable medium comprising one or more constituents of the solid flavor material entrained in the vapor or aerosol.

In some cases, the method comprises (i) volatilizing a liquid to form a vapor or aerosol, wherein the liquid comprises a liquid tobacco extract, and (ii) flowing the vapor or aerosol through a solid flavor material to form an inhalable medium, the inhalable medium comprising one or more constituents of the solid flavor material entrained in the vapor or aerosol.

A fifth aspect of the invention provides a liquid tobacco extract having a water activity of less than about 0.45 Aw at 25° C., for use as a volatilizable liquid in aerosol generating apparatus.

A sixth aspect of the invention provides a cartridge for use in an aerosol generating device, wherein the cartridge contains a liquid comprising a liquid tobacco extract, wherein the liquid tobacco extract is volatilized in use to form an inhalable vapor or aerosol. The liquid tobacco extract (a) has a water activity of less than about 0.45 Aw at 25° C. and/or (b) is that obtainable by or obtained by a process comprising

(i) extraction of tobacco components from tobacco using a supercritical extraction solvent, and (ii) transfer of the extracted tobacco components into a liquid solvent.

A seventh aspect of the invention provides an aerosol generating device, comprising a cartridge according to the sixth aspect of the invention, an atomizer and an outlet, the apparatus being configured such that the inhalable medium passes out of the outlet. In some cases, the cartridge may comprise the atomizer.

The invention also provides an aerosol generating device, the device comprising a liquid comprising a liquid tobacco extract, wherein the liquid tobacco extract is volatilizable in use to form an inhalable vapor or aerosol;

an atomizer for volatilizing the liquid; and an outlet;

the device being configured such that in use, the liquid is volatilized to form a vapor or an aerosol which passes out of the outlet;

characterized in that the liquid tobacco extract (a) has a water activity of less than about 0.45 Aw at 25° C. and/or (b) is that obtainable by or obtained by a process comprising (i) extraction of tobacco components from tobacco using a supercritical extraction solvent, and (ii) transfer of the extracted tobacco components into a liquid solvent.

The or each atomizer may be a heater, such as an electrically resistive heater. The heater may alternatively be an induction heater, a thin film heater, a blade heater etc. In some cases, the or each atomizer may be a piezoelectric device, such as that described in WO 2012/062600 (which is incorporated by reference in its entirety).

An eighth aspect of the invention provides a kit of parts, the kit comprising a cartridge according to the sixth aspect of the invention and a volatilizing device, wherein the cartridge is configured to be operable with the volatilizing device so as to generate an inhalable vapor or aerosol.

An aerosol generating device according to the sixth, seventh or eighth aspect of the invention may be an electronic cigarette, or may be an electronic tobacco hybrid device. Electronic cigarettes are described in U.S. Pat. Nos. 8,948,578 and 9,555,199 which are specifically incorporated by reference herein, in their entirety.

Further features and advantages of the invention will become apparent from the following description of preferred embodiments of the invention, given by way of example only, which is made with reference to the accompanying drawings.

### BRIEF DESCRIPTION OF THE DRAWINGS

FIGS. 1 and 2 show respectively a perspective view and a side elevation of an example of an apparatus for generating an inhalable medium;

FIGS. 3a to 3e show schematic longitudinal cross-sectional views of an example of a cartridge having a first chamber for containing liquid and an integral second chamber for solid flavor material and components thereof;

FIGS. 4a to 4e show schematic longitudinal cross-sectional views of an example of a cartridge having a first chamber for containing liquid and a separate second chamber for solid flavor material and components thereof and

FIGS. 5a to 5e show schematic longitudinal cross-sectional views of an example of a cartridge having a first chamber for containing liquid and a separate second chamber for solid flavor material and components thereof.

### DETAILED DESCRIPTION

It may be noted that, in general, a vapor is a substance in the gas phase at a temperature lower than its critical temperature, which means that for example the vapor can be condensed to a liquid by increasing its pressure without reducing the temperature. On the other hand, in general, an aerosol is a colloid of fine solid particles or liquid droplets, in air or another gas. A “colloid” is a substance in which microscopically dispersed insoluble particles are suspended throughout another substance. The terms “aerosol” and “vapor” are often used interchangeably in practice.

The first aspect of the invention provides a cartridge for use with an apparatus for generating an inhalable medium, the cartridge comprising:

a first chamber containing a liquid, the liquid comprising a liquid tobacco extract;

a second chamber configured to receive a solid flavor material; and

the cartridge being arranged such that in use, the liquid is volatilized to form a vapor or an aerosol, and the vapor or aerosol combines with one or more components of the solid flavor material received in the second chamber to form an inhalable medium, the inhalable medium comprising one or more constituents of the solid flavor material entrained in the vapor or aerosol.

In some cases, the cartridge additionally comprises a channel providing fluid communication between the first chamber and the second chamber, wherein in use, the aerosol or vapor formed by volatilizing the liquid passes through the channel to the second chamber and through the solid flavor material to form the inhalable medium. In this embodiment, the second chamber is arranged downstream of the first chamber.

In alternative embodiments, the second chamber may be arranged upstream of the first chamber, wherein volatilized components of the solid flavor material flow through the liquid in use. In yet further embodiments, the first and second chambers are not in fluid communication; the volatilized components of the liquid and solid flavor material flow out of the respective first and second chambers and are combined downstream of each chamber.

In some cases, the second chamber contains the solid flavor material.

In some cases, the solid flavor material comprises tobacco. In some cases, the solid flavor material consists essentially of or consists of tobacco. The tobacco may be any suitable solid tobacco, such as single grades or blends, cut rag or whole leaf, ground tobacco, tobacco fiber, cut tobacco, extruded tobacco, tobacco stem and/or reconstituted tobacco. The tobacco may be of any type including Virginia and/or Burley and/or Oriental tobacco.

The liquid in the first chamber may, in some cases, consist essentially of or consist of a liquid tobacco extract.

In some cases, the liquid may be volatilized in the first chamber. In other cases, it may be volatilized in the flow path downstream of the first chamber, such as, for example between the first and second chambers. Where the second chamber is arranged downstream of the first chamber, the liquid is typically volatilized to form a vapor or aerosol before it reaches the second chamber, so that the vapor or aerosol flows through the solid flavor material to entrain components of that material.

In some cases, the cartridge may have a single substantially tubular channel providing fluid communication between the first chamber and the second chamber. In other cases, the cartridge may have plural channels providing fluid communication between the first chamber and the second chamber. In yet further cases, the channel providing fluid communication between the first chamber and the second chamber may be an annular channel arranged so that it substantially surrounds the first chamber.

In some cases, the cartridge may comprise an atomizer which is arranged to volatilize the liquid in the first chamber in use. In some cases, the cartridge may comprise an atomizer which is arranged to volatilize the solid flavor material in the second chamber in use. The or each atomizer may be a heater, such as an electrically resistive heater. The heater may alternatively be an induction heater, a thin film heater, a blade heater etc.

In some cases, the or each atomizer may be a piezoelectric device, such as that described in WO 2012/062600 which is incorporated by reference in its entirety.

In some cases, the first and second chambers are spaced; that is, they are substantially not in conductive thermal contact. The distance between the first and second chambers may be the minimum sufficient to achieve physical separation of the chambers. The distance maybe up to 50 mm, preferably up to 25 mm, more preferably up to 15 mm. The inventors have found that spacing the chambers allows improved control over the heat profile experienced by each of the consumables (i.e. the liquid comprising a liquid tobacco extract and the solid flavor material). However, the spacing should not be too large in order to minimize the distances that vapors flow, thereby minimizing heat loss.

In some cases, the cartridge may comprise a wick for wicking liquid held in the first chamber out of the first chamber in use.

In some cases, the first and second chambers of the cartridge may be provided as an integral component. In other cases, the first and second chambers may be provided as separate components which are detachably connected to each other.

In some cases, the cartridge may be disposable or may be rechargeable with liquid tobacco extract and/or solid flavor medium.

The second aspect of the invention provides apparatus for generating an inhalable medium, the apparatus comprising a cartridge according to the first aspect of 30 the invention, and an outlet, the apparatus being configured such that the inhalable medium passes out of the outlet.

The invention also provides apparatus for generating an inhalable medium, the device comprising:

a first chamber containing a liquid, the liquid comprising a liquid tobacco extract;

a second chamber configured to receive a solid flavor material; and an atomizer for volatilizing the liquid in the first chamber; and

an outlet;

the apparatus being configured such that, in use, the liquid is volatilized to form a vapor or an aerosol, and the vapor or aerosol combines with one or more components of the solid flavor material received in the second chamber to form an inhalable medium which passes out of the outlet, the inhalable medium comprising one or more constituents of the solid flavor material entrained in the vapor or aerosol.

The features described above in relation to the cartridge of the first aspect may be incorporated into the apparatus of the second aspect accordingly to the extent that they are compatible.

In some cases, the apparatus additionally comprises a channel providing fluid communication between the first chamber and the second chamber, wherein in use, the aerosol or vapor formed by volatilizing the liquid passes through the channel to the second chamber and through the solid flavor material to form the inhalable medium.

In some cases, the second chamber contains the solid flavor material. In some cases, the solid flavor material comprises tobacco.

In some cases, the apparatus may additionally comprise an atomizer which is which is arranged to volatilize the solid flavor material in the second chamber in use.

The or each atomizer may be a heater, such as an electrically resistive heater.

The heater may alternatively be an induction heater, a thin film heater, a blade heater etc. In some cases, the or each atomizer may be a piezoelectric device, such as that described in WO 2012/062600 which is incorporated by reference in its entirety.

The apparatus for generating an inhalable medium may additionally comprise a battery component which provides power to the atomizer(s) in use. The apparatus for generating an inhalable medium may additionally comprise a mouthpiece.

### Description of the Apparatus Illustrated in the Figures

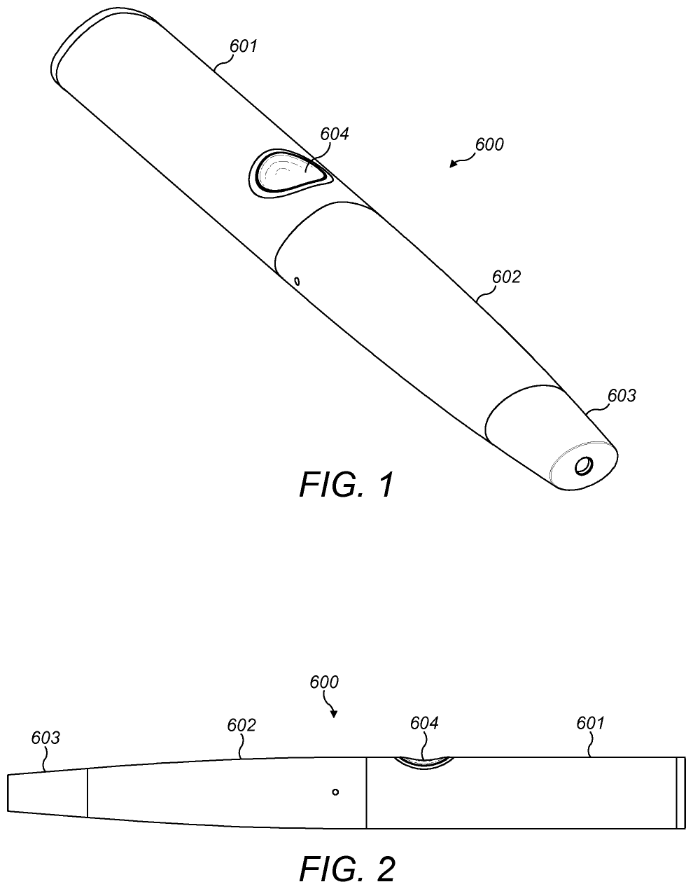

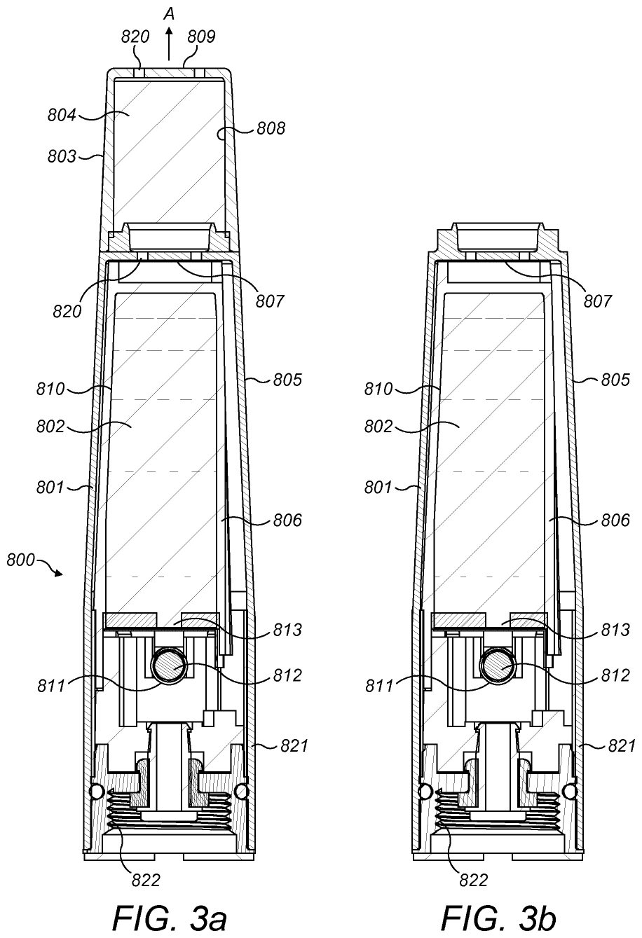

FIGS. 1 and 2 show respectively a perspective view and a side elevation of an example of an apparatus 600 for generating an inhalable medium. The apparatus 600 may be used with any of the cartridges described herein and with other cartridges. The apparatus 600 has a battery section 601 and a cartridge section 602. The battery section 601 and the cartridge section 602 are shown connected to each other in the drawings, but can be separated by a user to allow a cartridge to be loaded into the cartridge section 602. The battery section 601 and the cartridge section 602 can be separably connected to each other using for example a snap-fit connection, clips, a screw thread, etc. The cartridge section 602 has a mouthpiece 603 at its remote end. In this example, the battery section 601 has an on-off or power button 604. The battery section 601 contains a power supply, such as a battery which may be a rechargeable battery or a disposable battery. The battery section 601 also contains a controller for controlling the operation of various components of the apparatus 600 and/or a puff detector. In use, a user separates the battery section 601 and the cartridge section 602, inserts a cartridge into the cartridge section 602, and then connects the battery section 601 and the cartridge section 602 together again. The user can then operate the apparatus 600 using the on-off or power button 604.

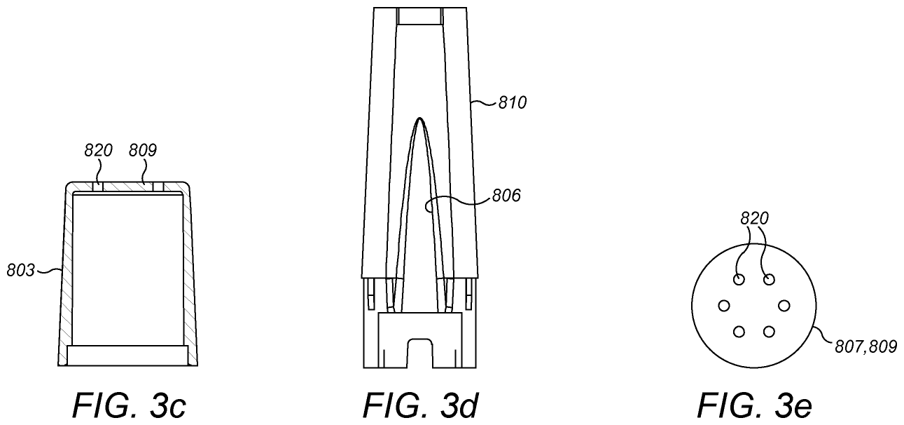

Referring now to FIGS. 3a to 3e, there is shown a schematic longitudinal cross-sectional view of an example of a cartridge 800 having a first chamber 801 for containing liquid 802 and a second chamber 803 for solid flavor material 804. In this example, the first chamber 801 and the second chamber 803 are provided as one integral component by being formed initially of two parts, shown separately in FIGS. 3c and 3d, which are then assembled as shown in FIG. 3a in a substantially permanent fashion. The first chamber 801 and the second chamber 803 may be fixed to each other by for example friction welding, such as spin welding, ultrasonic welding, etc. The cartridge 800 is arranged so that as the liquid 802 is volatilized so as to produce an aerosol of liquid droplets or sufficiently heated to produce a vapor, at least some and preferably all or substantially all of the aerosol or vapor passes through the material 804 to pick up flavor from the material 804.

In the examples of FIGS. 3a to 3e, the first chamber 801 shown is approximately cylindrical in shape. In other examples (not shown) the first chamber 801 may be frustoconical or approximately frustoconical, or may have a different shape, such as conical, and may have a circular or oval or polygonal cross-sectional shape, etc.

The first chamber 801 of the cartridge 800 has an outer shell 805 which defines a groove or channel 806 around the outside of the length of the inner liquid chamber part 810 and which extends from one end of the first chamber 801 to the other. In the example shown, this channel is provided by a groove 806 in the outer wall of the inner liquid chamber part 810, as can be seen most clearly in FIG. 3d. In other examples, there may be a plurality of channels 806 which extend from one end of the first chamber 801 to the other. In yet other examples, the channel 806 around the outside of the length of the inner liquid chamber part 810 may be an annular channel which passes around the whole of the inner liquid chamber part 810 and extends from one end of the first chamber 801 to the other.

The cartridge 800 has a heater 811 for heating liquid and a wick 812 in thermal contact with the heater 811. The heater 811 may be for example an electrically resistive heater, a ceramic heater, etc. In this example, the heater 811 and the wick 812 are provided as a single unit. In this case, where the cartridge 800 includes a heater 811, such a cartridge is often referred to as a “cartomizer”. The orientation of the heater 811 is shown schematically and for example the heater 811 may be a coil having its longitudinal axis parallel to the longitudinal axis of the cartridge 800 rather than perpendicular as shown in FIGS. 3a and 3b.

The wick 812 is in contact with the liquid 802. This may be achieved by for example the wick 812 being inserted through a through hole in the second end wall 813 of the inner liquid chamber part 810. Alternatively or additionally, the second end wall 813 may be a porous member which allows liquid to pass through from the inner liquid chamber part 810, and the wick 812 may be in contact with the porous second end wall 813. The second end wall 813 may be for example in the form of a porous ceramic disk. The wick 812 is generally absorbent and acts to draw in liquid 802 from the inner liquid chamber part 810 by capillary action. The wick 812 is preferably non-woven and may be for example a cotton or wool material or the like, or a synthetic material, including for example polyester, nylon, viscose, polypropylene or the like, or a ceramic material.

In use, the cartridge 800 is connected by the user to a battery section of an apparatus (which may for example be an apparatus 600 like that shown in FIGS. 1 and 2) to enable the liquid heater 811 to be powered. When the liquid heater 811 is powered (which may be instigated for example by the user operating a button 604 of the overall apparatus 600 or by a puff detector of the overall apparatus 600, as is known per se), liquid 802 drawn in from the inner liquid chamber part 810 by the wick 812 is heated by the heater 811 to volatilize or vaporize the liquid. As the user draws on a mouthpiece 603 of the overall apparatus 600, the vapor or aerosol passes into the channel 806 of the first chamber 801 and into the chamber 808 that contains the solid flavor material 804. The vapor or aerosol picks up flavor from the material 804. In the case that the material 804 contains or includes nicotine, the vapor or aerosol also contains nicotine entrained from the material 804. The vapor or aerosol can then exit the cartridge 800 as shown by the arrow A. A one way valve (not shown) may be provided so that the vapor or aerosol can only exit the cartridge 800 and cannot back-flow to the heater 811 or the electronics of the apparatus as a whole.

The chamber 808 for the solid flavor material 804 is closed off at the mouth end by an end wall 809 which is spaced from the end wall 807 of the first chamber 801. The end wall 809 of the chamber 808 may be provided by a separate retainer 809, for example in the form of a disk 809 which is inserted into the chamber 808 during manufacture. As an alternative, as in the example shown, the end wall 809 may be part of the second chamber 803. The end wall or retainer or disk 809 may be formed of plastics or rubber or ceramic or the like and has one or more through holes 820 to allow aerosol or vapor to pass through the mouth end of the cartridge 800.

Similarly, the end wall 807 of the first chamber 801 may be provided by a separate retainer 807, for example in the form of a disk 807 which is fitted to the first chamber 801 during manufacture, or, as in the example shown, the end wall 807 may be part of the first chamber 801. The disk 807 may be formed of plastics or rubber or ceramic or the like and has one or more through holes 820 to allow aerosol or vapor to pass into the second chamber 808 which contains the solid flavor material 804.

The end wall/disk 807 of the first chamber 801 and the end wall/disk 809 of the second chamber 808 assist in retaining the solid flavor material 804 in position in the second chamber 808, both during transport of the cartridge 800 and during use of the cartridge 800.

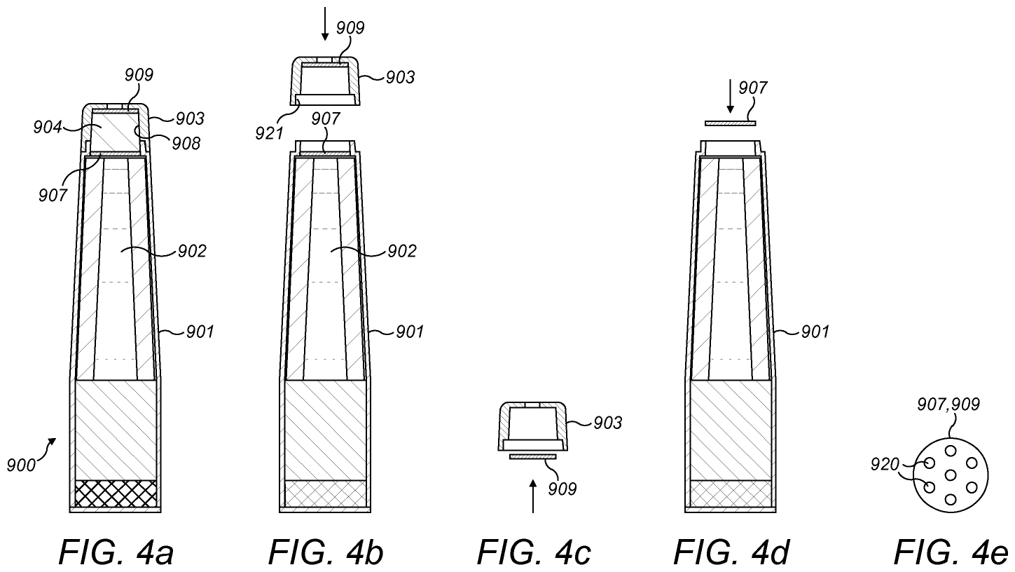

Referring now to FIGS. 4a to 4e, there is shown a further example of a cartridge 900 having a first chamber 901 for containing liquid 902 and a second chamber 903 for material 904, which will typically be solid flavor material 904. Many aspects and features of the example of FIGS. 4a to 4e are similar to the example described above with reference to of FIGS. 3a to 3e and a detailed description of those aspects and features will not be repeated here for the sake of brevity.

In this example, the first chamber 901 and the second chamber 903 are provided as separate parts, shown separately in FIGS. 4c and 4d. The first chamber 901 and the second chamber 903 may be connected or fixed to each other during manufacture, by for example clipping them together, by friction welding, such as spin welding, ultrasonic welding, etc. As an alternative, the first chamber 901 and the second chamber 903 may be connected or fixed to each other by the user during use, with the second chamber 903 being in the form of a removable end cap. In such a case, the user can easily replace one or other of the first chamber 901 and the second chamber 903 as required as the liquid 902 or solid flavor material 904 as the case may be is consumed during use. The second chamber 903 may be fitted to the second chamber 901 by for example clips and/or as a friction fit. The cartridge 900 is again arranged so that as the liquid 902 is volatilized so as to produce an aerosol of liquid droplets or sufficiently heated to produce a vapor, at least some and preferably all or substantially all of the aerosol or vapor passes through the material 904 to pick up flavor from the material 904.

The second chamber 908 for the solid flavor material 904 is closed off at the mouth end by an end wall 909. In the example shown the end wall 909 at the mouth end of the chamber 908 is provided by a separate disk 909 which is inserted into the chamber 908 during manufacture. The disk 909 may be formed of plastics or rubber or ceramic or the like and has one or more through holes 920 to allow aerosol or vapor to pass through the mouth end of the cartridge 900. As an alternative, the end wall 909 may be part of the second chamber 903, and similarly has one or more through holes to allow aerosol or vapor to pass through the mouth end of the cartridge 900 to be inhaled by a user. In a further alternative, the chamber 908 may have a second end wall or cap (not shown) having through holes, spaced from the end wall or disk 909 at the mouth end. In this way, the chamber 908 for the solid flavor material 904 can provide a complete unit that contains the solid flavor material 904, which facilitates manufacture of the overall cartridge 900 and/or simplifies replacement of the chamber 908 by the user once the solid flavor material 904 has been consumed.

Similarly, in the example shown, the end wall 907 of the first chamber 901 is provided by a separate disk 907 which is fitted to the first chamber 901 during manufacture. The disk 907 may be formed of plastics or rubber or ceramic or the like and has one or more through holes 920 to allow aerosol or vapor to pass into the chamber 908 which contains the solid flavor material 904. As an alternative, the end wall 907 may be part of the first chamber 901, and similarly has one or more through holes to allow aerosol or vapor to pass through. Either way, the first chamber 901 can be a complete, sealed unit that contains the liquid 902, which facilitates manufacture of the overall cartridge 900 and/or simplifies replacement of the first chamber 901 by the user once the liquid 902 has been consumed.

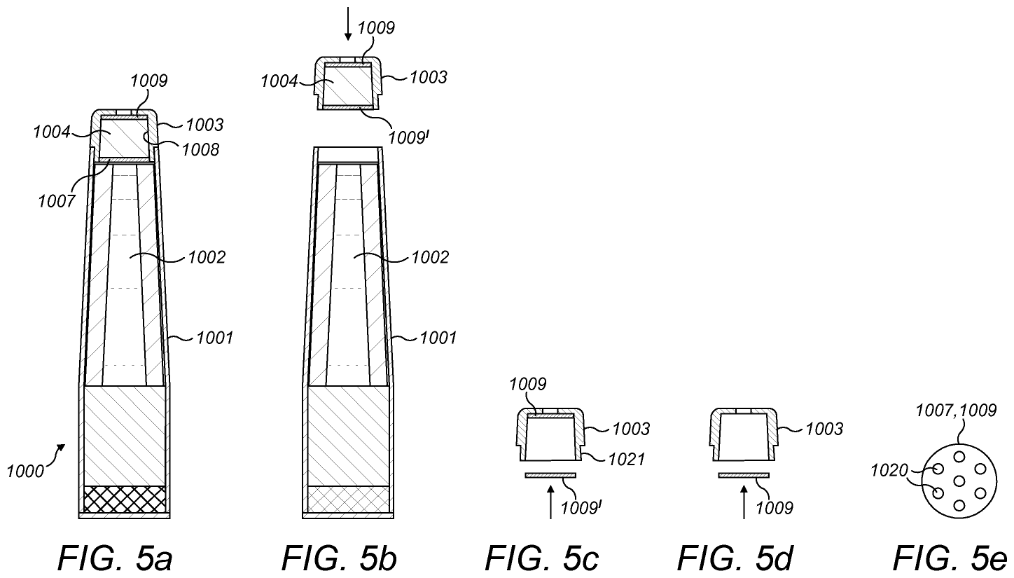

In the example of FIGS. 4a to 4e, the second chamber or end cap 903 is a female connector, having an annular end wall 921 which fits over the end of the first chamber 901 in use. In FIGS. 5a to 5e, there is shown another example cartridge 1000 having a first chamber 1001 for containing liquid 1002 and a second chamber 1003 for material 1004, which will typically be solid flavor material 1004. The example of FIGS. 5a to 5e is very similar to the example of FIGS. 4a to 4e except that in this case, the second chamber or end cap 1003 is a male connector, having an annular end wall 1021 which fits within the end of the first chamber 1001 in use. FIGS. 5a to 5e also show an example of the embodiment mentioned above in which the second chamber 1003 has a second end wall or cap 1009′ having through holes, spaced from the end wall or disk 1009 at the mouth end, so that the chamber 1008 for the solid flavor material 1004 provides a complete unit that contains the solid flavor material 1004.

The examples shown in FIGS. 3, 4 and 5 are particularly suitable for use with so-called modular products, in which the cartomizer is fitted to a battery section of an overall apparatus (such as a battery section 601 of an apparatus 600 as shown in FIGS. 1 and 2), typically by a screw thread, a bayonet fitting or the like. The cartomizer as a whole is typically discarded after use and a new, replacement cartomizer used. As an alternative, it may be possible for the user to re-use the cartridge by refilling the liquid and/or replacing the solid flavor material from time to time as necessary.

The examples shown in FIGS. 3, 4 and 5 may easily be adapted for use with other types of e-cigarette apparatus, which are known per se. There are for example so-called “look alike e-cigarette” or “cig-alike” devices which are generally small and have a form and appearance similar to a conventional cigarette. In such devices, the first chamber typically includes some wadding material, of for example cotton or the like, for holding the liquid. The cartridge or cartomizer in such known devices is typically disposable as a whole, but it may be possible to refill the liquid and/or replace the solid flavor material in examples that use an embodiment of the present invention.

Particularly in examples where it is possible to refill the liquid and/or replace the solid flavor material, the atomizer (such as a heater) may be a permanent part of the apparatus. As another example, there are so-called tank devices or personal vaporizers which generally have large liquid containers for holding relatively large volumes of liquid and also provide for advanced functions that allow users to control a number of aspects of the device.

As an alternative to any of the cartomizer arrangements discussed above, the atomizer (such as a heater) for the liquid may be provided separately of the liquid and material chambers. The atomizer may for example be provided as part of the battery section 601 of the overall apparatus 600 to which the cartridge is detachably fitted by the user in use.

In any of the examples described above in relation to FIGS. 3, 4 and 5, there may also be provided a atomizer for the solid flavor material so as to “pre-heat” the solid flavor material. This solid flavor material atomizer (such as a heater) may be provided as part of the cartridge or as part of the battery section of the apparatus to which the cartridge is fitted in use.

A number of other variations and alternatives to the examples described above are possible.

For example, in some cases it may be possible for the solid flavor material to be located, exclusively or additionally, in the mouthpiece of the apparatus (e.g. the mouthpiece 603 of an apparatus 600 as described above) which with the cartridge described above is used.

As another example, the solid flavor material may be omitted from the apparatus. This might be at the option of the user. This provides the user with more flexibility over the use of the cartridge as the user can use the cartridge as a classic “e-cigarette” device, only vaporizing liquid and not having the vapor or aerosol pass over or through solid flavor material, from time to time if they choose. This is particularly the case for the examples where the solid flavor material in the second chamber is replaceable by the user.

In some examples, the outer shell 805 of the first chamber may extend beyond the heater 811 and wick 812 to form a skirt 821 which in use fits around a part of the housing or battery section 601 of the apparatus 600 with which the cartridge 800, 900, 1000 is used. Such a skirt 821 is shown in particular for the example of FIGS. 3a to 3e. These figures also show an example of the screw thread 822 in the cartridge 800 for fitting the cartridge 800 to the battery section 601 of the apparatus 600 as mentioned above.

In some examples, the channel through which aerosol/vapor flows from the liquid heater to the solid flavor material is annular and completely surrounds the first chamber. In other examples, the channel is not annular and does not surround the first chamber 801. For example, in some examples, such as that shown in FIGS. 3a to 3e, there may be a single, substantially tubular channel or groove extending from the first chamber to the solid flavor material. As another example, there may be plural channels or grooves extending from the first chamber to the solid flavor material, one or more of which may be substantially tubular. Where there are plural channels, it is possible for the channels to be filled with or contain or lead to materials having different properties. For example, one channel may be filled with or contain or lead to a material that imparts a first flavor to the vapor or aerosol, a second channel may be filled with or lead to a material that imparts a second flavor to the vapor or aerosol, etc.

In the examples above, the first chamber and the solid flavor material/second chamber are arranged substantially in-line, along a longitudinal axis of the apparatus or cartridge. In other examples, the first chamber and the solid flavor material/second chamber are arranged so as to at least partially overlap in the longitudinal direction of the apparatus or cartridge; in such examples, the first chamber and the solid flavor material/second chamber may still be arranged generally in-line along the longitudinal axis of the apparatus or cartridge, or may be arranged side by side, or with one partially or completely inside the other. In yet other examples, the first chamber and the solid flavor material/second chamber are arranged concentrically (either with the first chamber inside the solid flavor material/second chamber or vice versa), and may be arranged to be entirely off-set with respect to each other along the longitudinal axis of the apparatus or cartridge, or overlapping, or one completely within the other.

As another specific example, the solid flavor material/second chamber is placed in at least one channel between the atomizer and the outlet, the channel at least partially overlapping with the first chamber in the longitudinal axis of the apparatus or cartridge. In other words, the vapor or aerosol flow channel goes past the first chamber and the material is located somewhere within the channel.

The cartridge may comprise a cooler or heat exchanger, and/or the apparatus with which the cartridge 800, 900, 1000 is used may comprise a cooler or heat exchanger. The material and the cooler in such an arrangement may be separate and spaced from each other. The cooler may be downstream of the liquid atomizer and upstream of the second chamber, the cooler or cooling zone being arranged to cool vaporized liquid to form an aerosol of liquid droplets which in use passes through material received in the chamber. The cooler may be arranged in effect to act as a heat exchanger, allowing for recovery of heat from the vapor. The recovered heat can be used for example to pre-heat the material and/or to assist in heating the liquid

The volatilizable liquid comprises a liquid tobacco extract and is discussed in more detail below. Typically, it the volatilizable liquid is volatilizable in the range of 100−300° C. or more particularly around 150-250° C.; these ranges are preferable as this helps to keep down the power consumption of the apparatus with which the cartridge is used. As noted above, the solid flavor material is a material that may be used to impart a flavor to the aerosol or vapor produced from the liquid as the aerosol or vapor passes through the material. The material may for example consist of or comprise tobacco. As the aerosol or vapor passes through and over the tobacco material, the hot aerosol or vapor entrains organic and other compounds or constituents from the tobacco material that lend tobacco its organoleptic properties, thus imparting the flavor to the aerosol or vapor as it passes to the mouthpiece. It will be understood however that materials other than tobacco may be used to impart different flavors to the aerosol or vapor stream. For example, flavorants could be included in the material or in the liquid.

In any of the examples described above, the apparatus controller controls operation of the apparatus as a whole. The controller for example may cause the or each heating element to be powered as and when required and switch off the or each heating element when heating is not required. Operation of the one or more heating elements may be controlled so that the liquid and/or material is heated to an optimum temperature. Particular considerations include ensuring that the solid flavor material does not burn, ensuring that adequate vaporization of the liquid is achieved, ensuring that the vaporized liquid or aerosol is at an appropriate temperature to liberate compounds from the solid flavor material, and ensuring that the vapor or aerosol that reaches the user is at a comfortable and safe temperature. A puff detector, a device which is known per se, may be provided to signal to the controller when the one or more heating elements need to be energized. The apparatus may also have one or more filters for filtering the vapor or aerosol before it reaches the user, cooling arrangements for cooling the vapor or aerosol before it reaches the user, insulation internally of the apparatus to protect the user from the heat generated inside the housing, etc.

In use, and particularly in the case that the material 804, 904, 1004 is or includes tobacco, it is preferred that the tobacco, or at least the surface of the tobacco, be heated to a temperature of between around 190° C. to 210° C. and most preferably around 200° C. so as to ensure that an adequate or appropriate amount of the compounds are released from the tobacco. As described in more detail above, the solid flavor material may be heated only by the hot vapor or aerosol that passes through the material or the solid flavor material may also be pre-heated or dual-heated using for example a dedicated heater. In the case of pre-heating, the solid flavor material, particularly in the case of tobacco, may be pre-heated to a temperature in the range of around 100 to 150° C. It will be appreciated however that other temperatures may be used. For example, the material, or at least the surface of the solid flavor material, may be heated to a temperature above 210° C., such as up to around 230° C. or 240° C. or so and even as high as 290° C. or so. The amount of tobacco present may be for example in the range 50 to 300 mg or so. A most suitable value for the amount of tobacco may be for example in the range 50 to 150 mg, with 130 mg being a value that is currently found to be particularly suitable in some applications. In a typical example, the amount of tobacco that is heated per operation of the apparatus (i.e. per puff) may be in the corresponding range of around 8 to 50 mg.

Suitable materials 804, 904, 1004 include materials that provide volatilized components upon heating, typically in the form of an aerosol. Suitable materials 804, 904, 1004 include any tobacco-containing material and may, for example, include one or more of tobacco per se, different varieties of tobacco, tobacco derivatives, expanded tobacco, reconstituted tobacco, ground tobacco, tobacco extract, homogenized tobacco or tobacco substitutes. In the case of tobacco, the material 804, 904, 1004 may be in the form of a rod of tobacco, a pod or plug of tobacco, loose tobacco, agglomerates, etc., and may be in relatively dry form or in relatively moist form for example. Suitable materials 804, 904, 1004 may include other, non-tobacco, products, which, depending on the product, may or may not contain nicotine.

In the particular case that the solid flavor material is tobacco, the tobacco may be in the form of a plug of tobacco rod which is cut to length and placed into the second chamber for the solid flavor material before the second chamber for the solid flavor material is combined with the first chamber (whether the second chamber for the solid flavor material is combined with the first chamber during manufacture or by the user in use).

In some examples, the second chamber for the solid flavor material is transparent, so that the user can see the contents (i.e. the solid flavor material) in use, which is appealing to some users. The tobacco rod may be formed using a transparent material as a wrapping material, again so that the user can see the tobacco. A particularly suitable material is “NatureFlex” (trade mark), a biodegradable film made from renewable raw materials by Innovia Films Limited.

As used herein, the terms “flavor” and “flavorant” refer to materials which, where local regulations permit, may be used to create a desired taste or aroma in a product for adult consumers. They may include extracts (e.g., licorice, hydrangea, Japanese white bark magnolia leaf, chamomile, fenugreek, clove, menthol, Japanese mint, aniseed, cinnamon, herb, wintergreen, cherry, berry, peach, apple, Drambuie, bourbon, scotch, whiskey, spearmint, peppermint, lavender, cardamom, celery, cascarilla, nutmeg, sandalwood, bergamot, geranium, honey essence, rose oil, vanilla, lemon oil, orange oil, cassia, caraway, cognac, jasmine, ylang-ylang, sage, fennel, piment, ginger, anise, coriander, coffee, or a mint oil from any species of the genus Mentha), flavor enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, oil, liquid, or powder.

### Liquid Tobacco Extract

As used herein, the term “liquid tobacco extract” refers to tobacco components dissolved in a solvent. The extract may be formed by the treatment of any suitable tobacco, such as single grades or blends, ground tobacco, cut rag or whole leaf, including Virginia and/or Burley and/or Oriental tobacco.

The solvent suitably comprises or consists of an aerosol generating agent. As used herein, an “aerosol generating agent” is an agent that promotes the generation of an aerosol on heating. An aerosol generating agent may promote the generation of an aerosol by promoting an initial vaporization and/or the condensation of a gas to an inhalable solid and/or liquid aerosol.

Suitable aerosol generating agents include, but are not limited to: a polyol such as sorbitol, glycerol, and glycols like propylene glycol or triethylene glycol; a non-polyol such as monohydric alcohols, high boiling point hydrocarbons, acids such as lactic acid, glycerol derivatives, esters such as diacetin, triacetin, triethylene glycol diacetate, triethyl citrate or myristates including ethyl myristate and isopropyl myristate and aliphatic carboxylic acid esters such as methyl stearate, dimethyl dodecanedioate and dimethyl tetradecanedioate. In some cases, the solvent comprises or consists of one or more polyols, suitably propylene glycol and/or glycerol.

The liquid tobacco extract may have a water activity of less than about 0.45 Aw at 25° C., for use as a volatilizable liquid in aerosol generating apparatus. Suitably, the water activity of the extract may be less than about 0.4 Aw at 25° C. or 0.35 Aw at 25° C.

(Water Activity Values Determined Using Aqualab Prewater Activity Meter, Utilizing the Dewpoint Method.)

A method of making a liquid tobacco extract comprises (i) extraction of tobacco components from tobacco using a supercritical extraction solvent, and (ii) transfer of the extracted tobacco components into a liquid solvent.

For example, a method of making a liquid tobacco extract may comprise;

(a) contacting tobacco with an extraction solvent such that tobacco components are extracted from the tobacco into the solvent, wherein the extraction solvent comprises a supercritical fluid;

separating residual tobacco solids from the extraction solvent containing tobacco components;

providing an entrapment solvent in a vessel, exposing the extraction solvent containing tobacco components to conditions in that vessel, wherein the conditions in the vessel are such that the extraction solvent is subcritical, thereby releasing the tobacco components from the extraction solvent, and wherein the entrapment solvent dissolves the tobacco components released from the extraction solvent.

In some cases, the liquid tobacco extract referred to herein is that obtainable by, or obtained by this method.

In some cases, the entrapment solvent comprises an aerosol generating agent. In some cases, the entrapment solvent consists essentially or of consists of one or more aerosol generating agents. In some cases, the entrapment solvent used in the above method comprises a polyol. In some cases, the entrapment solvent comprises glycerol and/or propylene glycol. In some cases, the entrapment solvent consists essentially of or consists of glycerol.

In some cases, the extraction solvent used in the above process comprises carbon dioxide. In some cases, the extraction solvent consists essentially of or consists of carbon dioxide.

The method may additionally comprise an initial step of adding water to the tobacco. The amount of water added may be from about 2% to about 20% based on the dry weight of tobacco, suitably from about 2%, 5% or 8% to about 12%, 15%, 18% or 20%. This pre-treatment with water increases the transfer of polar tobacco components (such as flavors) from the tobacco to the entrapment solvent.

In this extraction process, a higher pressure under supercritical conditions increases the solvent capacity and increases the efficiency of extraction. However, more energy is required to achieve and maintain higher pressures. Thus, the supercritical conditions may suitably be selected to balance these conflicting requirements. Where the supercritical fluid comprises carbon dioxide, the pressure at which extraction occurs may, in some cases, be from about 8 MPa, 10 MPa, 15 MPa, 20 MPa or 25 MPa to about 85 MPa, 70 MPa, 55 MPa, 40 MPa or 30 MPa, suitably from 8-85 MPa, 15-40 MPa or 20-30 MPa. In some cases, the pressure may be from about 10-16 MPa, suitably about 12 MPa, or may be from about 20-26 MPa, suitably about 23 MPa; the inventors have found that the concentration of TSNAs in the tobacco extract is lower when extraction is completed at these pressures. Where the supercritical fluid comprises carbon dioxide, the temperature at which extraction occurs may, in some cases, be from about 308K, 318K or 328K to about 473K, 430K, 390K or 350K, suitably from 308-473K, 308-430K, or 328-350K.

The transfer to subcritical conditions reduces the fluid density of the extraction solvent and consequently results in precipitation of the tobacco components. The conditions must be such that the pressure is below the critical pressure of the extraction solvent and/or the temperature is below the critical temperature of the extraction solvent. For thermal efficiency, the conditions are suitably such that the pressure is below the critical pressure of the extraction solvent while the temperature remains above the critical temperature.

The efficiency of separation of the tobacco components and the extraction solvent improves as the conditions move further below the critical point of the extraction solvent. However, the extraction solvent will typically be collected and stored after separation (requiring compression); in some cases, it may be recycled into an extraction chamber. Thus, the subcritical conditions are suitably not too far below the critical point to improve energy efficiency. The subcritical conditions are suitably selected to balance these conflicting requirements. Where the extraction solvent comprises carbon dioxide, the pressure at which extraction occurs may, in some cases, be from about 3 MPa, 4 MPa, 5 MPa or 5.5 MPa to about 7.3 MPa, 7 MPa, 6.5 MPa, 6 MPa, 5.5 MPa or 5 MPa, suitably from 3-7.3 MPa, or 4-6 MPa. Where the extraction solvent comprises carbon dioxide, the temperature of the subcritical conditions under 20 which separation occurs may, in some cases, be from about 280K, 300K, 320K or 330K to about 473K, 430K, 390K or 350K, suitably from 308-473K, 308-430K, or 328-350K.

### Example of Process for Making Liquid Tobacco Extract

Pre-extraction (pre-treatment of tobacco): Ground Virginia tobacco leaf of the particle size ranging from 355 μm to 3.5 mm was pre-treated by addition of water (10% of total tobacco weight). The mixture of tobacco and water was left for equilibration for 15 minutes post water addition (which is sufficient time for the water to be fully absorbed).

The pre-treated tobacco (1.2 kg plus 10 wt % water) was placed in a stainless steel extraction basket and the basket placed in an extraction vessel (5L autoclave). The basket was closed at its ends by sinter metal plates (pore diameter 100 μm, pressure drop across the plate is no more than 1 bar), which distributed the supercritical fluid at the entrance and prevented egress of solid particles at the exit. The use of the basket also allowed the fast charge and discharge of the extraction vessel. The basket was sealed against the extraction vessel wall in order to prevent flow of the supercritical fluid around it.

The extraction vessel was linked to a separation vessel by a transfer line. A pressure regulation valve was present in the line. Carbon dioxide may suitably be pumped through the system at the rate 5-23 kg/hr. In this case, carbon dioxide was pumped through the system at the rate of 10 kg/hr. 1.2 kg of glycerol was provided in the separation vessel. The extraction chamber was maintained at 26 MPa and 338K and the separation chamber was maintained at 4.5 MPa and 318K. Precipitation of the extract in the separation chamber was achieved by pressure and temperature reduction (from supercritical to subcritical conditions), which reduced the fluid density of the carbon dioxide and therefore the solvent-power of the CO2. The extracted tobacco components were collected in the glycerol at the bottom of the separator.

Gaseous CO2 exited the separator after passing through a liquid/gas divider (which removed any remaining liquid extract that was entrained in the gas). The CO2 was collected and recycled into the extraction chamber.

The process was run for three hours and then the CO2 flow was shut-off. The system was depressurized (to atmospheric conditions). The glycerol containing tobacco components was then drained from the separator vessel and weighed.

### Tobacco Extract Properties:

| | | | | | Water |
|---|---|---|---|---|---|
| Nicotine | Water | Viscosity | | Density | activity |
| (mg/g) | (wt %) | (Pa s) | pH | (g/cm³) | (Aw) |
| 7-11 | 7.5-9 | 0.23 | 7.9 | 1.24 | 0.25-0.3 |

The process was run 6 times. Where ranges are provided in the data table, all examples are encompassed in that range. Where a single value is provided, the value is the mean value.

Water activity values reported below were measured at 24.9-25.2° C. using the Aqualab Prewater Activity meter. The values were determined using the dewpoint method. Viscosity values reported below were measured at 25° C. using a Gemini Rheometer from Bohlin Instruments.

Similar results have been observed when using Oriental or Burley starting tobaccos. The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilized and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. A cartridge for use with an apparatus for generating an inhalable medium, the cartridge comprising:
a first chamber containing a liquid, the liquid comprising a liquid tobacco extract;
a second chamber configured to receive a solid flavor material; and
the cartridge being arranged such that in use, the liquid is volatilized to form a vapor or an aerosol, and the vapor or aerosol combines with one or more components of the solid flavor material received in the second chamber to form an inhalable medium, the inhalable medium comprising one or more constituents of the solid flavor material entrained in the vapor or aerosol.

2. The cartridge according to claim 1, further comprising a channel providing fluid communication between the first chamber and the second chamber, wherein in use, the aerosol or vapor formed by volatilizing the liquid passes through the channel to the second chamber and through the solid flavor material to form the inhalable medium.

3. The cartridge according to claim 1, wherein the second chamber contains the solid flavor material.

4. The cartridge according to claim 3, wherein the solid flavor material comprises tobacco.

5. The cartridge according to claim 1, further comprising an atomizer which is arranged to volatilize the liquid in the first chamber in use.

6. The cartridge according to claim 1, further comprising an atomizer which is arranged to volatilize the solid flavor material in the second chamber in use.

7. The cartridge according to claim 1, wherein the liquid tobacco extract comprises tobacco components dissolved in a solvent, and wherein the solvent comprises a polyol.

8. The cartridge according to claim 7, wherein the solvent comprises propylene glycol and/or glycerol.

9. The cartridge according to claim 1, wherein the liquid tobacco extract is produced by the process of:
(i) extraction of tobacco components from tobacco using a supercritical extraction solvent, and
(ii) transfer of the extracted tobacco components into a liquid solvent.

10. The cartridge according to claim 9, wherein the supercritical extraction solvent comprises carbon dioxide.

11. An apparatus for generating an inhalable medium, the apparatus comprising a cartridge according to claim 1, and an outlet, the apparatus being configured such that the inhalable medium passes out of the outlet.

12. An apparatus for generating an inhalable medium, the apparatus comprising:
a first chamber containing a liquid, the liquid comprising a liquid tobacco extract;
a second chamber configured to receive a solid flavor material; and
an atomizer for volatilizing the liquid in the first chamber; and
an outlet;
the apparatus being configured such that, in use, the liquid is volatilized to form a vapor or an aerosol, and the vapor or aerosol combines with one or more components of the solid flavor material received in the second chamber to form an inhalable medium which passes out of the outlet, the inhalable medium comprising one or more constituents of the solid flavor material entrained in the vapor or aerosol.

13. The apparatus according to claim 12, further comprising a channel providing fluid communication between the first chamber and the second chamber, wherein in use, the aerosol or vapor formed by volatilizing the liquid passes through the channel to the second chamber and through the solid flavor material to form the inhalable medium.

14. The apparatus of claim 12, wherein second chamber contains a solid flavor material.

15. A kit comprising a cartridge according to claim 1 and a volatilizing device, wherein the cartridge is configured to be operable with the volatilizing device so as to generate an inhalable medium.

16. A method of generating an inhalable medium, the method comprising
(i) volatilizing a liquid to form a vapor or aerosol, wherein the liquid comprises a liquid tobacco extract, and
(ii) combining the vapor or aerosol with components of a solid flavor material to form an inhalable medium, the inhalable medium comprising one or more constituents of the solid flavor material entrained in the vapor or aerosol.

17. The method according to claim 16, wherein the solid flavor material comprises tobacco.

18. A liquid tobacco extract having a water activity of less than about 0.45 Aw at 25° C.

19. The liquid tobacco extract according to claim 18, wherein the extract is obtainable by
(i) extraction of tobacco components from tobacco using a supercritical extraction solvent, and
(ii) transfer of the extracted tobacco components into a liquid solvent.

20. The liquid tobacco extract according to claim 19, wherein the extract is obtainable by
(i) extraction of tobacco components from tobacco using supercritical carbon dioxide, and
(ii) transfer of the extracted tobacco components into a polyol solvent.

21. A cartridge for use in an aerosol generating device, wherein the cartridge contains a liquid comprising a liquid tobacco extract, wherein the liquid tobacco extract is volatilizable in use to form an inhalable vapor or aerosol, characterized in that the liquid tobacco extract (a) has a water activity of less than about 0.45 Aw at 25° C. and/or (b) is that obtainable by or obtained by a process comprising (i) extraction of tobacco components from tobacco using a supercritical extraction solvent, and (ii) transfer of the extracted tobacco components into a liquid solvent.

22. An aerosol generating device, the device comprising a cartridge according to claim 21, an atomizer and an outlet, the apparatus being configured such that the inhalable vapor or aerosol passes out of the outlet.

23. An aerosol generating device, the device comprising
a liquid comprising a liquid tobacco extract, wherein the liquid tobacco extract is volatilizable in use to form an inhalable vapor or aerosol;
an atomizer for volatilizing the liquid; and an outlet;
the device being configured such that in use, the liquid is volatilized to form a vapor or an aerosol which passes out of the outlet;
characterized in that the liquid tobacco extract (a) has a water activity of less than about 0.45 Aw at 25° C. and/or (b) is that obtainable by or obtained by a process comprising (i) extraction of tobacco components from tobacco using a supercritical extraction solvent, and (ii) transfer of the extracted tobacco components into a liquid solvent.

24. A kit of parts, the kit comprising a cartridge according to claim 21 and a volatilizing device, wherein the cartridge is configured to be operable with the volatilizing device so as to generate an inhalable vapor or aerosol.
